(19) United States
(12) Patent Application Publication
(10) Pub. No.: US 2022/0370265 A1
(43) Pub. Date: Nov. 24, 2022

(54) Anti-permeable, anti-side leakage diapers, pull ups and training pants, and a production line for producing the same

(71) Applicant: Mingde ZHANG, FUJIAN (CN)

(72) Inventor: Mingde ZHANG, FUJIAN (CN)

(21) Appl. No.: 17/325,232

(22) Filed: May 20, 2021

(51) Int. Cl. A61F 13/494; A61F 13/15

(52) U.S. Cl. CPC A61F 13/49453; A61F 13/15764

(57) ABSTRACT

An anti-permeable, anti-side leakage diaper, adapted to be used in ordinary diapers, pull ups or training pants, contains an outer layer, an inner layer, an absorbent core in between, and an anti-leakage layer on an inner side surface of the outer layer; an outer surface of the inner layer is provided with anti-side leakage strips corresponding to two lengthwise sides of the absorbent core respectively; each of the anti-side leakage strips is fixedly connected to the inner layer at one side edge, and another side edge is a free side edge provided with an elastic band along a lengthwise direction thereof; an anti-permeable layer is laminated onto each of the anti-side leakage strips along a lengthwise direction thereof. A production line for producing the diaper is also disclosed.

### BACKGROUND OF THE INVENTION

The present invention relates to the technical field of diaper products for babies or adults, and more specifically relates to anti-side leakage diapers, pull ups and training pants, and a production line for producing the same.

Nowadays, types of diapers for babies or adults commonly seen in the market include pieces of diapers, and pant-type pull ups and training pants. Regardless of the type, the structure of a diaper usually comprises an outer layer, an inner layer, and an absorbent core between the outer layer and the inner layer. A PE film layer is also provided between the outer layer and the absorbent core. An outer surface of the inner layer of the diaper is provided with anti-side leakage strips corresponding to two lengthwise sides of the absorbent core respectively. Each of the anti-side leakage strips is fixed at one side edge, and another side edge of which is a free side edge and is provided with an elastic band along a lengthwise direction thereof. The free side edges of the anti-side leakage strips are oppositely configured, and two ends of each of the free side edges are fixed to the inner layer. Due to contraction of the elastic bands, the anti-side leakage strips of the diaper will tightly surround the upper thighs of the user during use, so as to achieve the effect of anti-side leakage. However, the anti-side leakage strips are usually made of non-woven fabrics which may only achieve liquid blocking effect for a very short period of time, therefore the anti-side leakage strips are in fact not anti-permeable. Side leakage often occurs when user lies on one side. Although many diaper products nowadays feature an anti-side leakage function, most of them cannot really survive side leakage tests simulating a user lying on one side. When user lies on one side, urine flows to one side and cannot be timely absorbed by the absorbent core. Urine not being timely absorbed will leak out from the anti-side leakage strip(s) of the diaper. Accordingly, further technical solutions are needed to solve the technical problem of side leakage in diapers.

### BRIEF SUMMARY OF THE INVENTION

An object of the present invention is to provide anti-permeable, anti-side leakage diapers, pull ups and training pants that can solve the problem of side leakage.

To achieve the above object, the present invention provides the following technical solutions: An anti-permeable, anti-side leakage diaper (which may be ordinary diapers, pull ups or training pants), comprises an outer layer, an inner layer, an absorbent core between the outer layer and the inner layer, and an anti-leakage layer laminated to an inner side surface of the outer layer; an outer surface of the inner layer is provided with anti-side leakage strips corresponding to two lengthwise sides of the absorbent core respectively; each of the anti-side leakage strips is fixedly connected to the inner layer at one side edge, and another side edge of each of the anti-side leakage strips is a free side edge and is provided with an elastic band along a lengthwise direction thereof; an anti-permeable layer is laminated onto each of the anti-side leakage strips along a lengthwise direction thereof.

The anti-permeable layer is an anti-permeable polyethylene (PE) film, and/or the anti-leakage layer is an anti-leakage PE film.

Each of the anti-side leakage strips is double layered comprising upper non-woven fabrics and lower non-woven fabrics; the anti-permeable layer is laminated in between the double layer.

One side of the anti-permeable layer is recessed from the free side edge of the corresponding anti-side leakage strip along a lengthwise direction of the free side edge to define a space between the anti-permeable layer and the free side edge of the anti-side leakage strip; the elastic band is disposed at an outer side edge of the space corresponding to an outer edge of the free side edge.

Another side of the anti-permeable layer aligns with said one side edge of the anti-side leakage strip fixedly connected to the inner layer.

A production line for producing the diaper, comprising an absorbent core transfer section, an inner layer transfer section, an outer layer transfer section, and an integrated transfer section that integrates the absorbent core transfer section, the inner layer transfer section and the outer layer transfer section to produce the diaper; characterized in that: the inner layer transfer section comprises an anti-side leakage strip processing section; the anti-side leakage strip processing section comprises an upper non-woven fabrics transfer device, a lower non-woven fabrics transfer device, an anti-permeable PE film stretching and transfer device, an adhesive spraying device; a roller pressing and lamination device, a cutter device and an output device.

The upper non-woven fabrics transfer device transfers the upper non-woven fabrics to the roller pressing and lamination device; the lower non-woven fabrics transfer device also transfers the lower non-woven fabrics to the roller pressing and lamination device such that the lower non-woven fabrics are positioned below the upper non-woven fabrics in the roller pressing and lamination device; the anti-permeable PE film stretching and transfer device also transfers the anti-permeable layer to the roller pressing and lamination device such that the anti-permeable layer is positioned between the upper non-woven fabrics and the lower non-woven fabrics in the roller pressing and lamination device; before the lower non-woven fabrics and the anti-permeable layer are transferred to the roller pressing and lamination device, the adhesive spraying device is configured corresponding to a laminating inner surface of the non-woven fabrics to spray adhesive on the laminating inner surface of the non-woven fabrics; after the roller pressing and lamination device laminates the upper non-woven fabrics, the anti-permeable layer, and the lower non-woven fabrics, the laminated product is transferred to the output device; the cutter device is positioned between the roller pressing and lamination device and the output device; said cutter device is positioned such that the laminated product comprising the upper non-woven fabrics, the anti-permeable layer and the lower non-woven fabrics is cut along a lengthwise direction thereof to produce a first anti-side leakage strip and a second anti-side leakage strip symmetrical to each other.

The anti-side leakage strip processing section also comprises a deviation corrective device; the deviation corrective device is positioned prior to the anti-permeable layer stretching and transfer device transferring the anti-permeable layer to the roller pressing and lamination device, so as to correct deviation of the anti-permeable layer.

The anti-permeable layer stretching and transfer device comprises, sequentially along a direction of transfer of the anti-permeable layer, an output shaft on which a roll of anti-permeable layer is mounted, a film pressing device, a multi-segment reciprocating stretching device, and a flattening output roller assembly.

The above technical solutions provide the following beneficial effects: Compared with existing diaper products, the anti-permeable, anti-side leakage diaper of the present invention additionally provides an anti-permeable layer on each of the anti-side leakage strips wherein the anti-permeable layer can be made of PE film or other materials that may achieve the intended technical effects, so as to better solve the problem of side leakage due to permeation in the technical solution of side leakage prevention described in the background. Structural features such as double layer, provision of spaces and alignments are provided such that the diaper of the present invention may prevent easy damage of the anti-permeable layer (PE film), thereby achieving beneficial effects such as better anti-permeation effect and increased air permeability of the diaper.

Compared with the prior arts, the production line of the diaper according to the present invention is additionally provided with an anti-side leakage strip processing section in the inner layer transfer section of the prior arts, so as to process, transfer, laminate, cut and output the layered materials of the anti-side leakage strips. Transfer and processing are performed orderly. Also, the anti-side leakage strip processing section can be structured additionally to an existing prior art production line, such that modification of the existing equipment is easy, and the capital input for replacing the existing equipment can be reduced. The structures of the product according to the present invention and the production line of the same are applicable to similar products intended for similar purposes. Besides common products like pull ups and training pants for babies, the present invention is also applicable to adult pants, adult diapers and period panties for women.

### BRIEF DESCRIPTION OF THE DRAWINGS

FIG. 1 is a schematic structural view of the diaper according to the present invention.

FIG. 2 is a layered structural view of an anti-side leakage strip of the diaper according to the present invention.

FIG. 3 is a schematic structural view of the anti-side leakage strip of the diaper according to the present invention.

FIG. 4 is a schematic structural view of an anti-side leakage strip processing section in a production line of the diaper of the present invention.

FIG. 5 is a schematic structural view of the upper non-woven fabrics, the anti-permeable layer, and the lower non-woven fabrics laminated together, and a partial cutting line by a cutter device in the production line of the diaper of the present invention.

### DETAILED DESCRIPTION OF THE INVENTION

To further explain the technical solutions provided by the present invention, the present invention is further described in detail below with reference to an embodiment.

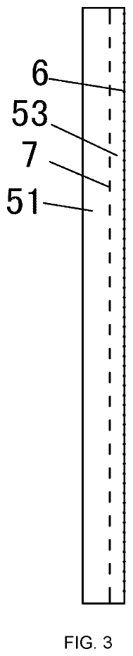

As shown in FIG. 1 to FIG. 3, a kind of anti-permeable, anti-side leakage diaper, which may be ordinary diapers, pull ups or training pants, comprises an outer layer 1, an inner layer 2, an absorbent core 3 between the outer layer 1 and the inner layer 2, and an anti-leakage layer 4 laminated to an inner side surface of the outer layer 1. The anti-leakage layer 4 is usually an anti-leakage polyethylene (PE) film, which is a commonly used material nowadays in the production of diaper products. An outer surface of the inner layer 2 of the diaper is provided with anti-side leakage strips 5 corresponding to two lengthwise sides of the absorbent core 3 respectively. Each of the anti-side leakage strips 5 is fixedly connected to the inner layer 2 at one side edge, and another side edge of which is a free side edge (such that the anti-side leakage strips 5 are configured such that they can be flipped or foldable with respect to the inner layer 2) and is provided with an elastic band 6 along a lengthwise direction thereof. Due to contraction of the elastic bands 6, the anti-side leakage strips 5 of the diaper will tightly surround the upper thighs of the user during use, so as to achieve the effect of anti-side leakage. Other structures of the diaper can be understood by reference to prior art products and will not be described in detail herein. The diaper of the present invention is different from prior art products mainly in that: an anti-permeable layer 7 is laminated onto each of the anti-side leakage strips 5 along a lengthwise direction thereof. The anti-permeable layer 7 can be an anti-permeable PE film made of PE. The provision of anti-permeable layers 7 can better block urine from leaking out of the diaper, and the urine retained in the diaper will be absorbed by the absorbent core. Accordingly, the intended object of the present invention can be achieved. The anti-permeable layers 7 can also be made of other non-harmful (to the user) and environmental friendly materials that achieve the same technical effects.

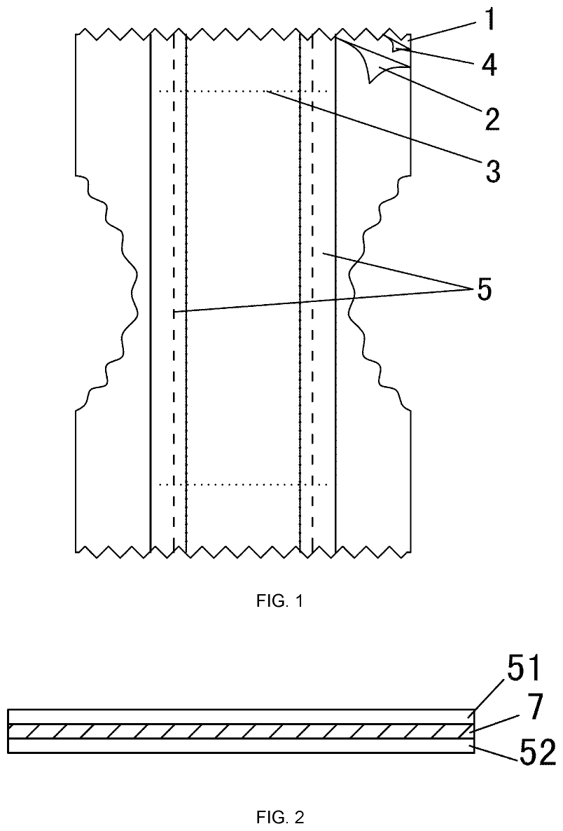

According to an embodiment and as shown in the figures, each of the anti-side leakage strips 5 is double layered comprising upper non-woven fabrics 51 and lower non-woven fabrics 52. The anti-permeable layer 7 (PE film) is laminated in between the double layer as shown in FIG. 2 so as to prevent the anti-permeable layer 7 from damages, thereby attaining better anti-permeable effect. Also, one side of the anti-permeable layer 7 is recessed from the free side edge of the corresponding anti-side leakage strip 5 along a lengthwise direction of the free side edge to define a space 53 between the anti-permeable layer 7 and the free side edge of the anti-side leakage strip 5. As shown in the figures, the elastic band 6 is disposed at an outer side edge of the space 53 corresponding to an outer edge of the free side edge. Provision of the space 53 can increase air permeability of the diaper at the sides of the diaper. Provision of the elastic band 6 at the outer side edge of the space 53 can prevent the anti-permeable layer 7 from being damaged when assembling the elastic band 6 to the anti-side leakage strip during production and when the elastic band 6 is stretched during use, thereby preserving the anti-permeation effect of the anti-permeable layer 7. Further, another side of the anti-permeable layer 7 aligns with said one side edge of the anti-side leakage strip 5 fixedly connected to the inner layer 2 so that production is easier and a better anti-permeation effect can be achieved at said one side edge of the anti-side leakage strip.

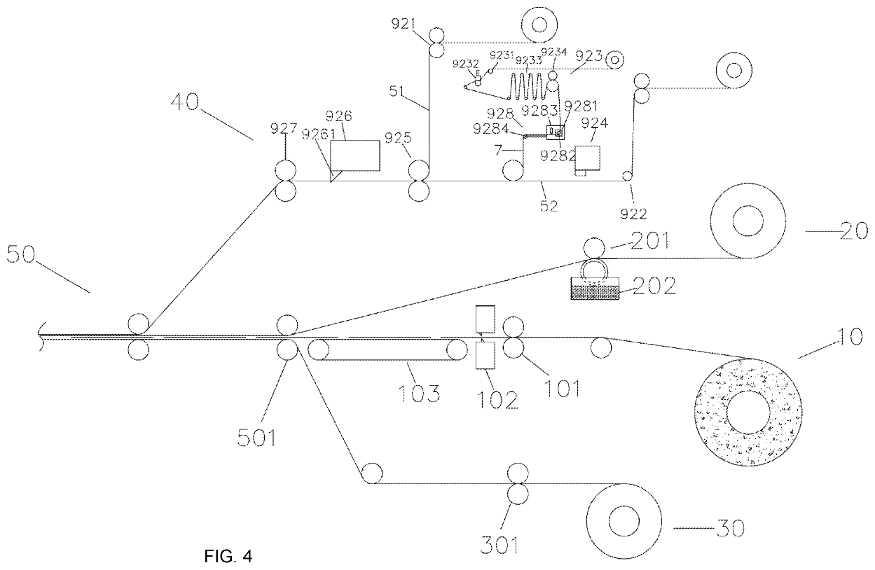

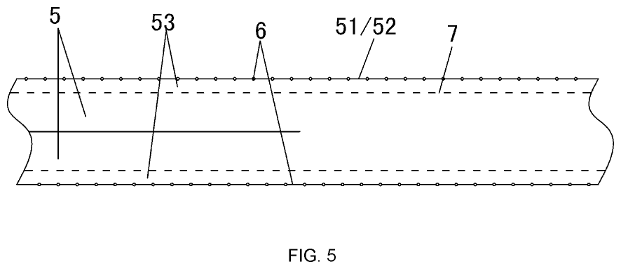

A production line for producing the diaper is disclosed below. Since the novel and inventive feature of the diaper of the present invention is mainly the structural configuration of the anti-side leakage strips 5, the production line of the present invention is different from the prior art production line mainly in the additional processing steps of the anti-side leakage strips and additional production equipment correspondingly required. According to an embodiment, the production line for producing the diaper comprises an absorbent core transfer section 10, an inner layer transfer section 20, an outer layer transfer section 30, and an integrated transfer section 50 that integrates the absorbent core transfer section 10, the inner layer transfer section 20 and the outer layer transfer section 30 to produce the diaper. The absorbent core transfer section 10 is disposed between the inner layer transfer section 20 and the outer later transfer section 30, and specifically the inner layer transfer section 20 is located above the absorbent core transfer section 10, and the outer layer transfer section 30 is located below the absorbent core transfer section 10. The absorbent core production section, the inner layer processing section, the outer layer processing section, and the integrated production section are prior arts obtained from a prior art production line, wherein according to FIG. 4, the absorbent core transfer section 10 comprises a roll of prepared absorbent cores produced according to the prior art, a pair of core transmission rollers 101 that transfers the absorbent cores, a cutter assembly 102 provided subsequent to the core transmission rollers 101 to cut the transferred absorbent cores from the roll of prepared absorbent cores separated absorbent cores, and a transmission belt 103 provided subsequent to the cutter assembly 102 to transfer the cut absorbent cores output from the cutter assembly 102 to the integrated transfer section 50. The inner transfer section 20 comprises a roll of prepared inner layers produced according to the prior art, a pair of inner layer transmission rollers 201 that transfers the inner layers from the roll of prepared inner layers to the integrated transfer section 50, a glue supply 202 disposed below a lower roller of the pair of inner layer transmission rollers 201 such that when the inner layer transmission rollers 201 rotates, a surface of the lower roller is coated with glue from the glue supply 202, and thus applying the glue to the lower surfaces of the transferred inner layers before the inner layers are transferred to the integrated transfer section 50 wherein the transferred inner layers are positioned above the absorbent cores. The outer transfer layer 30 comprises a roll of outer layers prepared according to prior art, a pair of outer layer transmission rollers 301 that transfers the outer layers to the integrated transfer section 50 where the transferred outer layers are positioned below the integrated transfer section 50. The integrated transfer section 30 comprises a pair of press rollers 501 that press the materials transferred from said absorbent core transfer section 10, the inner layer transfer section 20 and the outer layer transfer section 30 against one another. Since it is understood that the absorbent core transfer section 10, the inner layer transfer section 20 and the outer layer transfer section 30 as well as the prepared absorbent cores, the prepared inner layers, and the prepared outer layers transferred by these sections 10, 20, 30 are prior arts, any detailed structures thereof that may not be fully described herein should be known to a person skilled in the art and should not affect the understanding of the present invention. An embodiment of the present invention should focus on describing the processing of the anti-side leakage strips in the inner layer transfer section. The inner layer transfer section comprises an anti-side leakage strip processing section 40; the anti-side leakage strip processing section 40 comprises an upper non-woven fabrics transfer device 921, a lower non-woven fabrics transfer device 922, an anti-permeable PE film stretching and transfer device 923, an adhesive spraying device 924; a roller pressing and lamination device 925, a cutter device 926 and an output device 927.

The upper non-woven fabrics transfer device 921, which comprises a pair of transmission rollers, transfers the upper non-woven fabrics 51 to the roller pressing and lamination device 925; the lower non-woven fabrics transfer device 922, which also comprises a pair of transmission rollers, also transfers the lower non-woven fabrics 52 to the roller pressing and lamination device 925 such that the lower non-woven fabrics are positioned below the upper non-woven fabrics in the roller pressing and lamination device; the anti-permeable PE film stretching and transfer device 923 also transfers the anti-permeable layer (PE film) to the roller pressing and lamination device 925 such that the anti-permeable layer is positioned between the upper non-woven fabrics 51 and the lower non-woven fabrics 52 in the roller pressing and lamination device 925; before the lower non-woven fabrics 52 and the anti-permeable layer (PE film) are transferred to the roller pressing and lamination device 925, the adhesive spraying device 924, which is a prior art standard device which is commonly available in the market for producing diapers, is configured corresponding to a laminating inner surface of the non-woven fabrics 52 to spray adhesive on the laminating inner surface of the non-woven fabrics 52. After the roller pressing and lamination device 925, which comprises a pair of press rollers disposed oppositely and rotate in mutually opposite directions, laminates the upper non-woven fabrics 51, the anti-permeable layer (PE film), and the lower non-woven fabrics 52, the laminated product will be transferred to the output device 927 which comprises a pair of rollers that output the laminated product. The cutter device 926 is positioned between the roller pressing and lamination device 925 and the output device 927; in other words, after the upper non-woven fabrics 51, the anti-permeable layer 7 and the lower non-woven fabrics 52 are laminated, and before the laminated product is transferred to the output device 927, the laminated product is being cut by the cutter device 926 before being transferred to the output device 927; said cutter device 926, which comprises a cutter knife 9261, is positioned such that the laminated product comprising the upper non-woven fabrics 51, the anti-permeable layer 7 and the lower non-woven fabrics 52 is cut along a lengthwise direction along a center lengthwise axis thereof to produce a first anti-side leakage strip (for left side of the diaper) and a second anti-side leakage strip (for right side of the diaper) symmetrical to each other. In the present embodiment, the laminated product is cut and divided so as to produce the first and second anti-side leakage strips simultaneously. By using the above described configurations in the production line, structural arrangements of the above devices according to the present embodiment are simpler, and the production efficiencies of the same are enhanced.

The anti-side leakage strip processing section also comprises a deviation corrective device 928; the deviation corrective device 928 is positioned before the anti-permeable layer (PE film) is transferred to the roller pressing and lamination device 925 by the anti-permeable layer stretching and transfer device 923, so as to correct deviation of the anti-permeable layer (PE film). The deviation corrective device 928 ensures that the anti-permeable layer (PE film) aligns with the upper non-woven fabrics 51 and the lower non-woven fabrics 52 between the upper non-woven fabrics 51 and the lower non-woven fabrics 52 when the anti-permeable layer (PE film) is laminated together with the upper non-woven fabrics 51 and the lower non-woven fabrics 52. The deviation correction device 928 comprises left and right adjustment shafts 9282 whose ends are connected with a driving cylinder 9281, sensor heads 9283 located corresponding to two sides of the anti-permeable layer (PE film), and an output roller 9284; when the anti-permeable layer stretching and transfer device 923 transfers the anti-permeable layer (PE film), the anti-permeable layer (PE film) passes through the left and right adjustment shafts 9282 and being transferred to the output roller 9284 before it is eventually transferred to the roller pressing and lamination device 925. The use of sensor heads 9283 sense a position of the anti-permeable layer (PE film) passing through the left and right adjustment shafts 9282 so that the driving cylinder 9281 can be controlled to adjust the left and right adjustment shafts 9282 to adjust the position of the anti-permeable layer (PE film) in case of deviation of the anti-permeable layer (PE film), thereby achieving deviation correction.

The anti-permeable layer stretching and transfer device 923 comprises, sequentially along a direction of transfer of the anti-permeable layer (PE film), an output shaft 9231 on which a roll of anti-permeable layer (PE film) is mounted, a film pressing device 9232 comprising a press block and a press roller correspondingly disposed against each other, a multi-segment reciprocating stretching device 9233 comprising multiple shafts staggered with respect to one another, and a flattening output roller assembly 9234; the film pressing device 9232 can unify a thickness of the anti-permeable layer (PE film) prior to outputting the anti-permeable layer; the multi-segment reciprocating stretching device 9233 stretches the anti-permeable layer evenly so that the anti-permeable layer reaches an optimal stretched condition; the flattening output roller assembly 9234 can flatten the stretched anti-permeable layer before transferring the same to the roller pressing and lamination device 925 such that during lamination, the anti-permeable layer is flatly laminated.

The anti-side leakage strip processing section is structurally configured such that it can be additionally configured into a prior art production line by modifying the prior art production line. Therefore, the present invention can be possibly achieved without making any irreversible changes to prior art production equipment. Therefore, the present invention has the advantages of easy modification of the existing equipment and reduced capital input of altering and replacing the existing equipment.

The above embodiment and drawings are not intended to limit the form and type of the present invention. Any appropriate changes or modification of the present invention by a person skilled in this field of art should not be considered deviated from the scope of the present invention.

What is claimed is:

1. An anti-permeable, anti-side leakage diaper, comprising an inner layer, an absorbent core between the outer layer and the inner layer, and an anti-leakage layer laminated to an inner side surface of the outer layer; an outer surface of the inner layer is provided with anti-side leakage strips corresponding to two lengthwise sides of the absorbent core respectively; each of the anti-side leakage strips is fixedly connected to the inner layer at one side edge, and another side edge of each of the anti-side leakage strips is a free side edge and is provided with an elastic band along a lengthwise direction thereof; characterized in that: an anti-permeable layer is laminated onto each of the anti-side leakage strips along a lengthwise direction thereof.

2. The diaper of claim 1, wherein the anti-permeable layer is an anti-permeable polyethylene (PE) film, and/or the anti-leakage layer is an anti-leakage PE film.

3. The diaper of claim 1, wherein each of the anti-side leakage strips is double layered comprising upper non-woven fabrics and lower non-woven fabrics; the anti-permeable layer is laminated in between the double layer.

4. The diaper of claim 3, wherein one side of the anti-permeable layer is recessed from the free side edge of the corresponding anti-side leakage strip along a lengthwise direction of the free side edge to define a space between the anti-permeable layer and the free side edge of the anti-side leakage strip; the elastic band is disposed at an outer side edge of the space corresponding to an outer edge of the free side edge.

5. The diaper of claim 4, wherein another side of the anti-permeable layer aligns with said one side edge of the anti-side leakage strip fixedly connected to the inner layer.

6. A production line for producing the diaper as in claim 5, comprising an absorbent core transfer section, an inner layer transfer section, an outer layer transfer section, and an integrated transfer section that integrates the absorbent core transfer section, the inner layer transfer section and the outer layer transfer section to produce the diaper; characterized in that: the inner layer transfer section comprises an anti-side leakage strip processing section; the anti-side leakage strip processing section comprises an upper non-woven fabrics transfer device, a lower non-woven fabrics transfer device, an anti-permeable PE film stretching and transfer device, an adhesive spraying device; a roller pressing and lamination device, a cutter device and an output device;
the upper non-woven fabrics transfer device transfers the upper non-woven fabrics to the roller pressing and lamination device; the lower non-woven fabrics transfer device also transfers the lower non-woven fabrics to the roller pressing and lamination device such that the lower non-woven fabrics are positioned below the upper non-woven fabrics in the roller pressing and lamination device; the anti-permeable PE film stretching and transfer device also transfers the anti-permeable layer to the roller pressing and lamination device such that the anti-permeable layer is positioned between the upper non-woven fabrics and the lower non-woven fabrics in the roller pressing and lamination device; before the lower non-woven fabrics and the anti-permeable layer are transferred to the roller pressing and lamination device, the adhesive spraying device is configured corresponding to a laminating inner surface of the non-woven fabrics to spray adhesive on the laminating inner surface of the non-woven fabrics; after the roller pressing and lamination device laminates the upper non-woven fabrics, the anti-permeable layer, and the lower non-woven fabrics, the laminated product is transferred to the output device; the cutter device is positioned between the roller pressing and lamination device and the output device; said cutter device is positioned such that the laminated product comprising the upper non-woven fabrics, the anti-permeable layer and the lower non-woven fabrics is cut along a lengthwise direction thereof to produce a first anti-side leakage strip and a second anti-side leakage strip symmetrical to each other.

7. The production line of claim 6, wherein the anti-side leakage strip processing section also comprises a deviation corrective device; the deviation corrective device is positioned prior to the anti-permeable layer stretching and transfer device transferring the anti-permeable layer to the roller pressing and lamination device, so as to correct deviation of the anti-permeable layer.

8. The production line of claim 6, wherein the anti-permeable layer stretching and transfer device comprises, sequentially along a direction of transfer of the anti-permeable layer, an output shaft on which a roll of anti-permeable layer is mounted, a film pressing device, a multi-segment reciprocating stretching device, and a flattening output roller assembly.
